(19) United States
(12) Patent Application Publication
(10) Pub. No.: US 2023/0071100 A1
(43) Pub. Date: Mar. 9, 2023

(54) DEVICE OF GENERATING INACTIVATED VIRUSES AND KILLING RESPIRATORY VIRUSES IN EXHALED AIR IN REAL TIME AND METHOD THEREOF

(71) Applicants: Tzong-Shi Chiueh, Taipei City (TW); Wan-Ying Lin, Taoyuan City (TW)

(72) Inventors: Tzong-Shi Chiueh, Taipei City (TW); Wan-Ying Lin, Taoyuan City (TW)

(21) Appl. No.: 17/900,996

(22) Filed: Sep. 1, 2022

(30) Foreign Application Priority Data
Sep. 7, 2021 (TW) 110133135

(51) Int. Cl. A61M 16/10; A61M 16/06; A62B 18/02; A61L 2/10

(52) U.S. Cl. CPC A61M 16/1065; A61M 16/0683; A62B 18/025; A61L 2/10

(57) ABSTRACT

A device of generating inactivated viruses and killing respiratory viruses in exhaled air in real time includes a mask; a wearable assist member having two ends secured to the mask; and a filter attached to the mask, the filter including a rear exhaled air collection chamber, an intermediate member for deactivating viruses, and a front chamber for passing air with inactivated viruses all communicating with each other. The rear exhaled air collection chamber includes an inlet attached to a front portion of the mask and communicating therewith, and a space communicating with the inlet. The intermediate member for deactivating viruses includes a channel and at least one element for deactivating viruses. The front chamber for passing air with inactivated viruses includes at least one outlet on a front end and an internal fan spaced from the at least one outlet.

### FIELD OF THE INVENTION

The invention relates to deactivating viruses and more particularly to a device of generating inactivated viruses and killing respiratory viruses in exhaled air in real time and method thereof in which when a patient wears the device, viruses in exhaled air are deactivated and respiratory viruses in the exhaled air are killed in real time by the device and antigenicity of viruses of the exhaled air are not damaged, and thus the exhaled air is safe to discharge into the atmosphere.

### BACKGROUND OF THE INVENTION

Currently, measure for preventing respiratory virus infection is either quarantine or vaccine protection. Once a person is infected, the person (i.e., patient) is required to be quarantined. Also, all persons in close contact with the patient are listed as ones at risk. The patient may suffer great pressure when the patient is in an isolation ward for treatment. Protection is implemented by continuously drawing air out of the ward in negative atmospheric pressure. Further, a filter is used to filter out airborne viruses at exhalation. It can greatly consume both software and hardware of medical resources. Furthermore, more medical employees are needed to take care of the patient. For a medical employee, wearing a protective gown when enters the ward and taking off the protective gown when leaving the ward are required. This is time consuming. In short, a great amount of resources are consumed for treating the patient and preventing viruses from spreading.

Traditionally, a great number of viruses are required to culture and next the viruses are deactivated by means of chemicals or any of other methods. However, there is safety concern for highly contagious viruses. Further, not all viruses can be used for making vaccines from itself because the making of vaccines from viruses is very difficult. Regarding vaccines made from inactivated viruses, the vaccines are not effective because selected virus strains for making vaccines have antigens different from the current strains. In other words, it is required to confirm type of antigen or type of genome prior to making vaccines from viruses so that the selected virus strains for making vaccines have antigens the same as the current strains. However, antigenic variants are great due to high evolution of virus genomes. There are escaped strains in produced vaccines and in turn, effectiveness of the vaccines is decreased or even ineffective. Moreover, a high dose of antigens in the conventional vaccines is injected into the muscle. In addition, a variety of excipients are used in the injection. This is not a natural method of injecting respiratory viruses into the human body. After a vaccine has been injected into the body, side effects including pain, fever, etc. in the muscle where the vaccine is injected may be felt by the person.

Thus, the need for improvement still exists.

### SUMMARY OF THE INVENTION

It is therefore one object of the invention to provide a device of generating inactivated viruses and killing respiratory viruses in exhaled air in real time, comprising a mask; a wearable assist member having two ends secured to the mask; and a filter attached to the mask, the filter including a rear exhaled air collection chamber, an intermediate member for deactivating viruses, and a front chamber for passing air with inactivated viruses all communicating with each other wherein the rear exhaled air collection chamber includes an inlet attached to a front portion of the mask and communicating therewith, and a space communicating with the inlet; the intermediate member for deactivating viruses includes a channel and at least one element for deactivating viruses; and the front chamber for passing air with inactivated viruses includes at least one outlet on a front end and an internal fan spaced from the at least one outlet.

It is another object of the invention to provide a method of generating inactivated viruses and killing respiratory viruses in exhaled air in real time, the method comprising the steps of (a) wearing a mask on the nose and the mouth of a patient wherein the mask is attached to a filter and communicates therewith, and the filter includes a rear exhaled air collection chamber, an intermediate member for deactivating viruses, and a front chamber for passing air with inactivated viruses all communicating with each other; (b) flowing air having contagious viruses exhaled from both the mouth and the nose of the patient to the rear exhaled air collection chamber through the mask; (c) flowing the exhaled air from the rear exhaled air collection chamber to the intermediate member for deactivating viruses wherein the intermediate member for deactivating viruses includes at least one element for deactivating viruses, and the at least one element for deactivating viruses deactivates contagious viruses in the exhaled air in real time so that the exhaled air is free of contagious viruses and antigenicity of viruses of the exhaled air are not damaged; and (d) flowing the exhaled air with inactivated viruses from the intermediate member for deactivating viruses to the front chamber for passing air with inactivated viruses prior to discharging into the atmosphere.

By utilizing the device and the method, a patient having a respiratory tract infection wears the mask of the invention, air exhaled by the patient may have contagious viruses, the air flows through the mask to enter the filter which deactivate the viruses of the air, and the air having inactivated viruses is discharged into the atmosphere by the fan. As a result, the exhaled air is virus free and safe to be inhaled by persons in contact with the patient.

The invention has the following advantages and benefits in comparison with the conventional art:

Antigenicity of viruses of the exhaled air are not damaged. Viruses in the exhaled air are deactivated in real time. Current strains can be generated and can be spread into the environment by means of natural inhalation. There is no risk when a person contacts the patient wearing the mask of the device. Thus, the person is protected against being infected by viruses.

The device has a wide applications including being worn by a patient being infected by respiratory viruses. By wearing the device, there is no risk of the wearer being infected by viruses. Further, the inactivated viruses in the exhaled air discharged to the atmosphere can be used to make vaccines. There is no need to verify kind of contagious viruses. It is complied with current strains of pandemic. Contagious viruses of air exhaled by the patient are deactivated in real time. Thus, the exhaled air is free of contagious viruses.

The method of vaccine injection of the invention provides not only natural inhalation but also continuousness. The contagious viruses in the air exhaled by the patient are deactivated prior to discharging into the ward in real time. Thus, there is no risk for relatives visiting the patient or medical employees who care for the patient. Protection against viruses is well done when natural inhalation occurs. Herd immunity can be fulfilled. There is no need of quarantine. Hardware cost, work flow, consumed resources and burden of medical employees all are greatly decreased.

The above and other objects, features and advantages of the invention will become apparent from the following detailed description taken with the accompanying drawings

### BRIEF DESCRIPTION OF THE DRAWINGS

FIG. 1 is a perspective view of a device of generating inactivated viruses and killing respiratory viruses in exhaled air in real time according to a first preferred embodiment of the invention;

FIG. 2 is an exploded view in part section of the device:

FIG. 3 is a side elevation in part section of the device covered the nose and the mouth of a user in which the element for deactivating viruses is implemented as an ultraviolet screen device;

FIG. 4 is a front view of FIG. 3 with the mask removed viewing from the exhaled air collection chamber to the ring shaped member for deactivating viruses;

FIG. 5 is a view similar to FIG. 3 where the element for deactivating viruses is implemented as a permeable substrate having chemicals for deactivating viruses;

FIG. 6 is a front view of FIG. 5 with the mask removed viewing from the exhaled air collection chamber to the ring shaped member for deactivating viruses;

FIG. 7 is a view similar to FIG. 3 where the element for deactivating viruses is implemented as a permeable substrate including nanoparticles having photothermal effect and at least one miniature light source is provided on an inner surface of the ring shaped member for deactivating viruses;

FIG. 8 is a front view of FIG. 7 with the mask removed viewing from the exhaled air collection chamber to the ring shaped member for deactivating viruses;

FIG. 9 is a view similar to FIG. 3 wherein the element for deactivating viruses is implemented as a UV screen device in conjunction with a permeable substrate including nanoparticles having photothermal effect and at least one miniature light source is provided on an inner surface of the ring shaped member for deactivating viruses;

FIG. 10 is a front view of FIG. 9 with the mask removed viewing from the exhaled air collection chamber to the ring shaped member for deactivating viruses;

FIG. 11 is an environmental view showing the nose and the mouth of the user covered by the device;

FIG. 12 is an environmental view showing the nose and the mouth of the user covered by a device according to a second preferred embodiment of the invention; and

FIG. 13 is a flow chart illustrating a method of generating inactivated viruses and killing respiratory viruses in exhaled air in real time according to the invention.

### DETAILED DESCRIPTION OF THE INVENTION

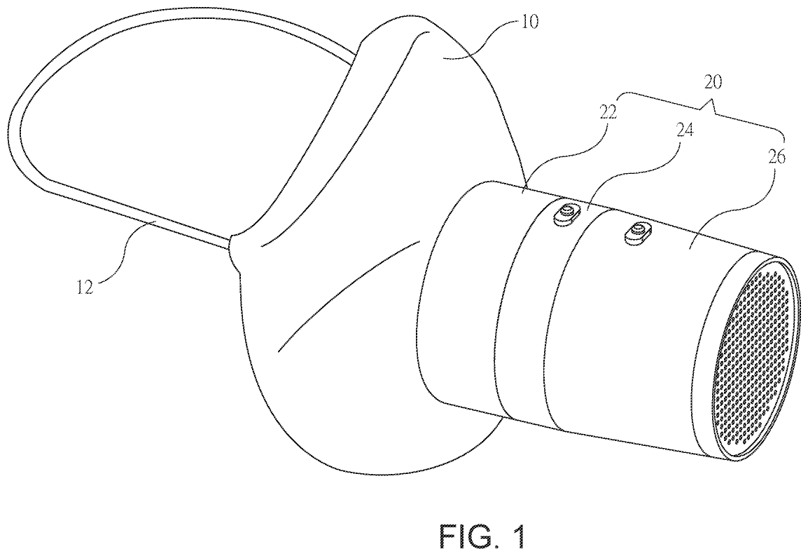

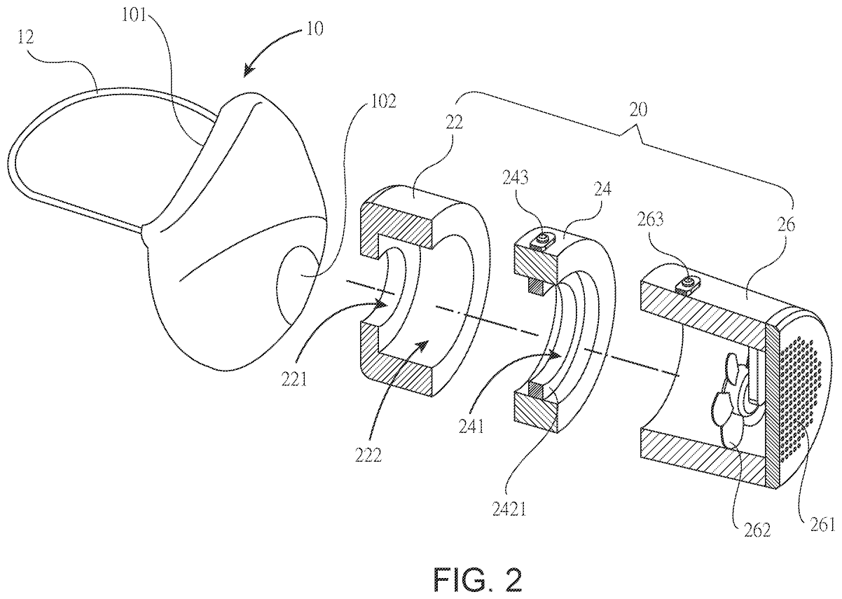

Referring to FIGS. 1 to 2, a device of generating inactivated viruses and killing respiratory viruses in exhaled air in real time according to a first preferred embodiment of the invention is shown and comprises a mask 10 and a filter 20 as discussed in detail below.

The mask 10 comprises a covering member 101 for covering the nose and the mouth of a user, and a front opening 102 communicating with inside of the mask 10. A wearable assist member 12 has two ends secured to the covering member 101. The filter 20 comprises a rear, cylindrical exhaled air collection chamber 22, an intermediate ring shaped member 24 for deactivating viruses, and a front, cylindrical chamber 26 for passing air with inactivated viruses all communicating with each other. The exhaled air collection chamber 22 is attached to a front portion of the mask 10 and includes an inlet 221 communicating with the opening 102. The exhaled air collection chamber 22 further comprises a space 222 communicating the inlet 221. The ring shaped member 24 for deactivating viruses includes a channel 241, at least one element 2421 for deactivating viruses, and a first control device 243 disposed on an outer surface and electrically connected to the element 2421 for deactivating viruses. The chamber 26 for passing air with inactivated viruses includes a multi-hole outlet 261 on a front end (i.e., distal the ring shaped member 24 for deactivating viruses), an internal fan 262 spaced from the outlet 261, and a second control device 263 disposed on an outer surface and electrically connected to the fan 262.

A patient may wear the device of the invention. Air exhaled by the patient passes through the mask 10 and the filter 20 which serve as a replacement of the typical negative pressure isolation room. The filter 20 can deactivate viruses in real time. As a result, the exhaled air is without contagious viruses and safe to discharge into the atmosphere.

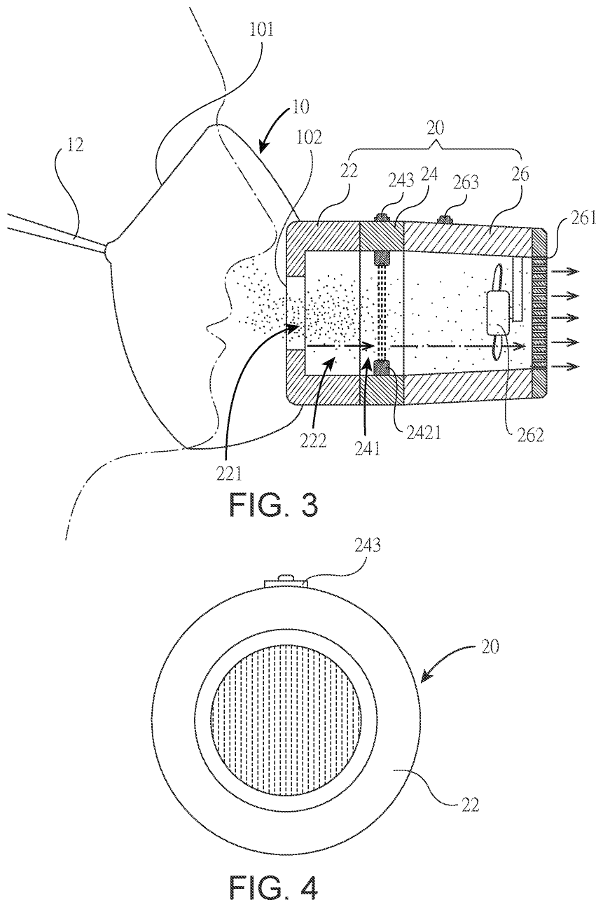

Referring to FIGS. 3 to 4, the element 2421 for deactivating viruses is implemented as an ultraviolet (UV) screen device and disposed on an inner surface of the ring shaped member 24 for deactivating viruses. Viewing from the exhaled air collection chamber 22 to the ring shaped member 24 for deactivating viruses, the UV screen device is in the channel 241. The UV screen device can be open or closed by activating the first control device 243 or not. After the UV screen device has been activated, UV light is emitted by the UV screen device and fills the channel 241. The UV light can deactivate viruses and thus the exhaled air is full of inactivated viruses, i.e., being not contagious. Further, the fan 262 creates a flow of the exhaled air and finally the airflow leaves the outlet 261 to discharge into the atmosphere.

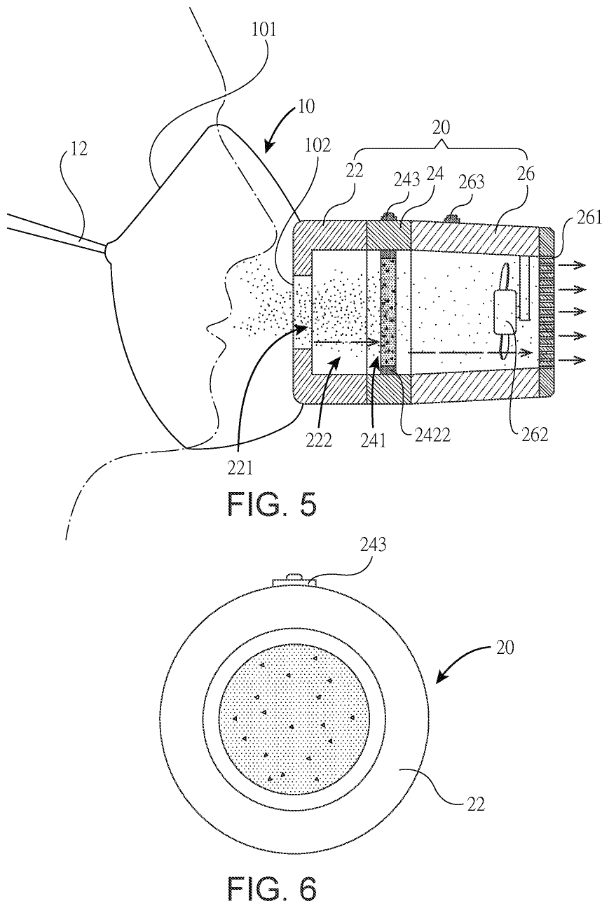

Referring to FIGS. 5 to 6, an element 2422 for deactivating viruses is implemented as a permeable substrate having chemicals for deactivating viruses and the chemicals are Triton X-100, Tween 80, tri(n-butyl) phosphate (TNBP), hypochlorous acid-containing water, ethylene glycol, riboflavin or amotosalen(4′-(4-amino-2-oxa)butyl-4,5′,8′-trimethylpsoralen). The channel 241 is filled with the element 2422 for deactivating viruses. The element 2422 for deactivating viruses can deactivate passing viruses.

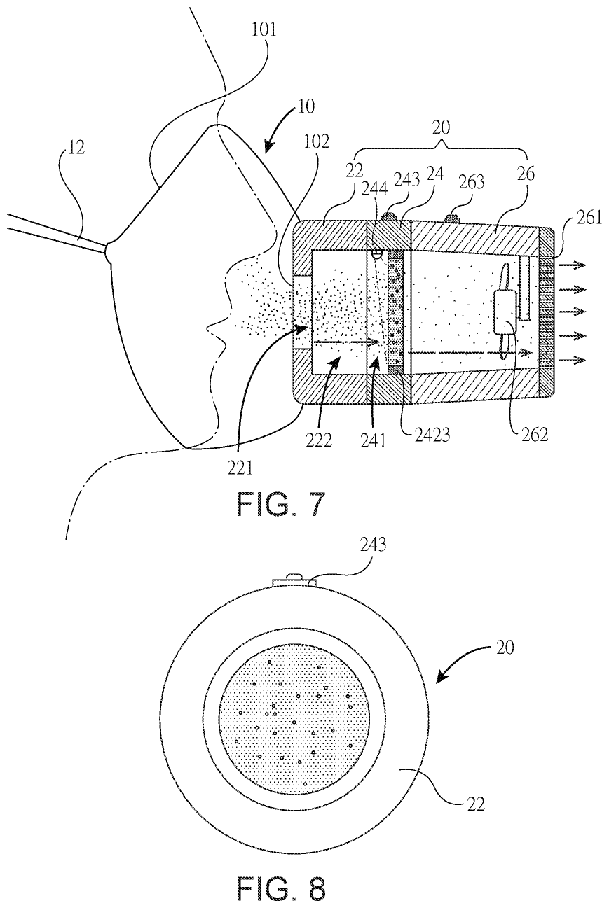

Referring to FIGS. 7 to 8, an element 2423 for deactivating viruses is implemented as a permeable substrate including nanoparticles having photothermal effect and fills the channel 241. At least one miniature light source 244 is provided on an inner surface of the ring shaped member 24 for deactivating viruses. On or off of the miniature light source 244 is controlled by activating the first control device 243 or not. Light emitted by the miniature light source 244 is directed to the element 2423 for deactivating viruses. After the miniature light source 244 has been turned on, light emitted by the miniature light source 244 is directed to the element 2423 for deactivating viruses. The nanoparticles of the element 2423 for deactivating viruses are excited to increase temperature of the element 2423 for deactivating viruses to 80° C.-95° C. The element 2423 for deactivating viruses can deactivate passing viruses by heat.

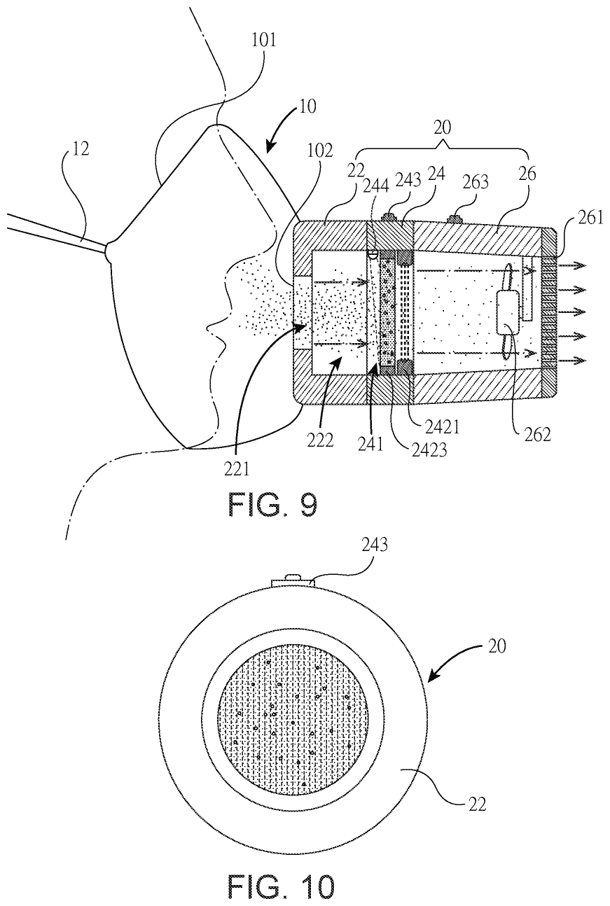

Referring to FIGS. 9 to 10, the elements 2421 and 2423 for deactivating viruses are provided in the channel 241; at least one miniature light source 244 is provided on the inner surface of the ring shaped member 24 for deactivating viruses. On or off of the miniature light source 244 is controlled by activating the first control device 243 or not. Light emitted by the miniature light source 244 is directed to the element 2423 for deactivating viruses which is implemented as a permeable substrate including nanoparticles having photothermal effect. The element 2421 for deactivating viruses is provided on the inner surface of the ring shaped member 24 for deactivating viruses and is controlled by activating the first control device 243 or not. After the UV screen device has been activated, emitted UV light fills the channel 241. Viruses passing the ring shaped member 24 for deactivating viruses can be deactivated by both the UV light and the heat.

Alternatively, the arrangement of the elements 2421, 2422 and 2423 for deactivating viruses shown in FIGS. 9 and 10 can be changed to one of many different combinations as long as viruses passing the ring shaped member 24 for deactivating viruses can be deactivated by the UV light, the heat and the permeable substrate having chemicals for deactivating viruses. This is a multi-level processing for completely deactivating viruses.

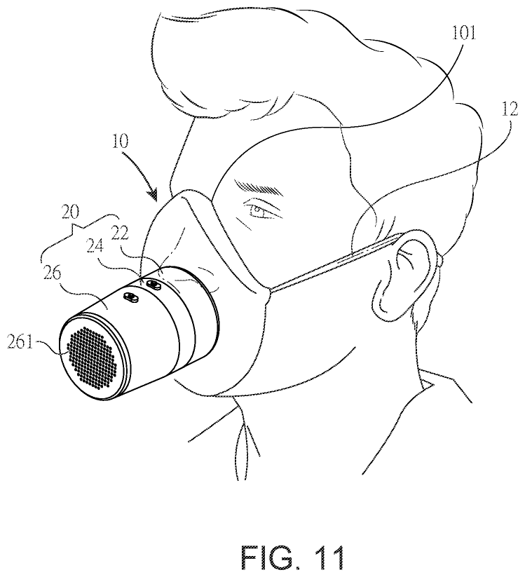

Referring to FIG. 11 in conjunction with FIGS. 1 to 2, a patient may wear the wearable assist member 12 on the head to fully attach the covering member 101 to the face. Air exhaled by the patient passes the mask 10 to enter the filter 20. Contagious viruses are deactivated by the filter 20. Finally, virus free air leaves the outlet 261.

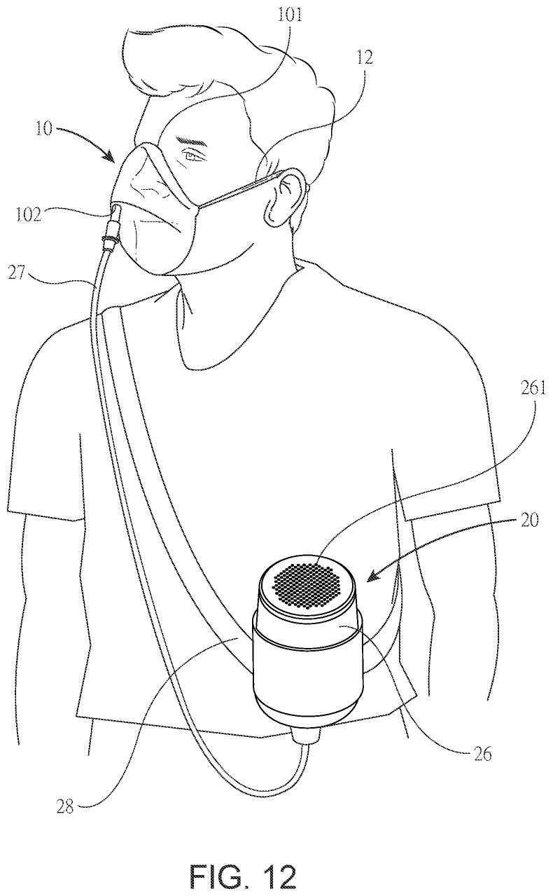

Referring to FIG. 12 in conjunction with FIG. 2, a device according to a second preferred embodiment of the invention is shown. The mask 10 comprises a covering member 101 for covering the nose and the mouth of a user, and a front opening 102 communicating with inside of the mask 10. A tube 27 has one end connected to the opening 102 and the other end connected to the exhaled air collection chamber 22. A sling 28 has two ends secured to the filter 20 and is draped across the torso of a user and over one shoulder of the user. The filter 20 is much larger than that of the first preferred embodiment which is designed to cover only the nose and the mouth of the user.

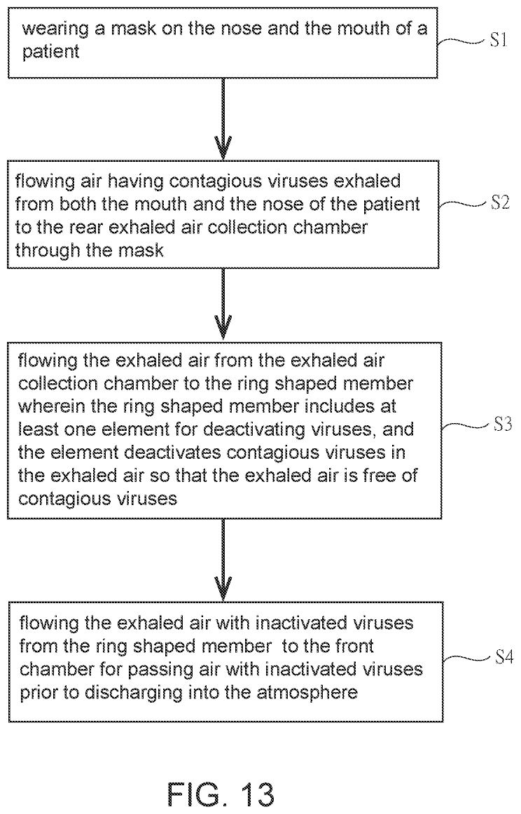

Referring to FIG. 13 in conjunction with FIGS. 1 to 2, a method of generating inactivated viruses and killing respiratory viruses in exhaled air in real time according to the invention is illustrated. The method comprises the steps of S1 wearing a mask 10 on the nose and the mouth of a patient in which the mask 10 is attached to a filter 20 and communicates therewith, and the filter 20 includes a rear, cylindrical exhaled air collection chamber 22, an intermediate ring shaped member 24 for deactivating viruses, and a front, cylindrical chamber 26 for passing air with inactivated viruses all communicating with each other; S2 flowing air having contagious viruses exhaled from both the mouth and the nose of the patient to the exhaled air collection chamber 22 through the mask 10; S3 flowing the exhaled air from the exhaled air collection chamber 22 to the intermediate ring shaped member 24 for deactivating viruses in which the intermediate ring shaped member 24 for deactivating viruses includes at least one element 2421 for deactivating viruses, and the at least one element 2421 for deactivating viruses deactivates contagious viruses in the exhaled air in real time so that the exhaled air is free of contagious viruses and antigenicity of viruses of the exhaled air are not damaged; and S4 flowing the exhaled air with inactivated viruses from the intermediate ring shaped member 24 for deactivating viruses to the chamber 26 for passing air with inactivated viruses prior to discharging into the atmosphere in which the at least one element 2421 for deactivating viruses is an UV screen device, a permeable substrate including nanoparticles having photothermal effect, a permeable substrate having chemicals for deactivating viruses, or any combination thereof.

While the invention has been described in terms of preferred embodiments, those skilled in the art will recognize that the invention can be practiced with modifications within the spirit and scope of the appended claims.

What is claimed is:

1. A device of generating inactivated viruses and killing respiratory viruses in exhaled air in real time, comprising:
a mask;
a wearable assist member having two ends secured to the mask; and
a filter attached to the mask, the filter including a rear exhaled air collection chamber, an intermediate member for deactivating viruses, and a front chamber for passing air with inactivated viruses all communicating with each other wherein the rear exhaled air collection chamber includes an inlet attached to a front portion of the mask and communicating therewith, and a space communicating with the inlet; the intermediate member for deactivating viruses includes a channel and at least one element for deactivating viruses; and the front chamber for passing air with inactivated viruses includes at least one outlet on a front end and an internal fan spaced from the at least one outlet.

2. The device of claim 1, wherein the at least one element for deactivating viruses is an ultraviolet (UV) screen device, a permeable substrate including nanoparticles having photothermal effect, a permeable substrate having chemicals for deactivating viruses, or any combination thereof; further comprising a first control device disposed on an outer surface of the intermediate member for deactivating viruses and electrically connected to the at least one element for deactivating viruses, and a second control device disposed on an outer surface of the front chamber for passing air with inactivated viruses and electrically connected to the internal fan.

3. The device of claim 2, wherein the at least one element for deactivating viruses is the UV screen device, and UV light emitted by the UV screen device fills the channel.

4. The device of claim 2, wherein the at least one element for deactivating viruses is the permeable substrate including nanoparticles having photothermal effect filled the channel; further comprising at least one miniature light source disposed on an inner surface of the intermediate member for deactivating viruses wherein on or off of the at least one miniature light source is controlled by activating the first control device or not, and light emitted by the miniature light source is directed to the at least one element for deactivating viruses so that after the at least one miniature light source has been turned on, light emitted by the at least one miniature light source is configured to excite the at least one element for deactivating viruses to increase temperature of the at least one element for deactivating viruses to 80° C.-95° C.

5. The device of claim 2, wherein the at least one element for deactivating viruses is the permeable substrate having chemicals for deactivating viruses filled the channel.

6. The device of claim 2, wherein the chemicals are Triton X-100, Tween 80, tri(n-butyl) phosphate (TNBP), hypochlorous acid-containing water, ethylene glycol, riboflavin or amotosalen (4′-(4-amino-2-oxa)butyl-4,5′,8′-trimethylpsoralen).

7. The device of claim 1, wherein the mask includes a covering member for covering the nose and the mouth of a user, and a front opening attached to the inlet of the rear exhaled air collection chamber and communicating therewith.

8. The device of claim 1, wherein the mask includes a covering member for covering the nose and the mouth of a user, and a front opening; further comprising a tube having one end connected to the front opening and the other end connected to the rear exhaled air collection chamber, and a sling having two ends secured to the filter.

9. A method of generating inactivated viruses and killing respiratory viruses in exhaled air in real time, the method comprising the steps of:
(a) wearing a mask on the nose and the mouth of a patient wherein the mask is attached to a filter and communicates therewith, and the filter includes a rear exhaled air collection chamber, an intermediate member for deactivating viruses, and a front chamber for passing air with inactivated viruses all communicating with each other;
(b) flowing air having contagious viruses exhaled from both the mouth and the nose of the patient to the rear exhaled air collection chamber through the mask;
(c) flowing the exhaled air from the rear exhaled air collection chamber to the intermediate member for deactivating viruses wherein the intermediate member for deactivating viruses includes at least one element for deactivating viruses, and the at least one element for deactivating viruses deactivates contagious viruses in the exhaled air in real time so that the exhaled air is free of contagious viruses and antigenicity of viruses of the exhaled air are not damaged; and
(d) flowing the exhaled air with inactivated viruses from the intermediate member for deactivating viruses to the front chamber for passing air with inactivated viruses prior to discharging into the atmosphere.

10. The method of claim 9, wherein the at least one element for deactivating viruses is an UV screen device, a permeable substrate including nanoparticles having photothermal effect, a permeable substrate having chemicals for deactivating viruses, or any combination thereof.
